(19) United States
(12) Patent Application Publication
(10) Pub. No.: US 2016/0310007 A1
(43) Pub. Date: Oct. 27, 2016

(54) Infrared light imaging apparatus

(71) Applicant: SHIMADZU CORPORATION, Kyoto-shi (JP)

(72) Inventor: Shinichiro ISHIDA, Kyoto-shi (JP)

(21) Appl. No.: 15/106,171

(22) PCT Filed: Dec. 18, 2013
(86) PCT No.: PCT/JP2013/083886

§ 371 Date: Jun. 17, 2016

(51) Int. Cl. A61B 5/00; A61M 5/00

(52) U.S. Cl. CPC A61B 5/0071; A61B 5/0091; A61B 5/418; A61B 5/4887; A61B 5/742; A61B 5/0077; A61M 5/007

(57) ABSTRACT

An infrared light imaging apparatus includes: a main body 10 including an input unit 11 such as a touch panel; an illumination/imaging unit 12 movably supported by an arm 13; a pair of display units 14 and 15; and a treatment table 16 on which a patient 17 is placed. The illumination/imaging unit 12 includes: a camera capable of detecting infrared rays and visible light; an infrared light source disposed in the outer circumferential part of the camera; and a visible light source disposed in the outer circumferential part of the infrared light source. On the display unit 14, a current fusion image of an affected area and its surroundings of the patient 17 is displayed as a moving image, whereas on the display unit 15, a past fusion image of the affected area and its surroundings of the patient 17 is displayed as a still image.

### TECHNICAL FIELD

This invention relates to an infrared light imaging apparatus for displaying an infrared image of a subject on a display unit.

### BACKGROUND ART

In recent years, a method called near-infrared fluorescence imaging has been utilized for surgery. In the near-infrared fluorescence imaging, indocyanine green (ICG) as a fluorescent dye is injected into an affected area. Then, when irradiating the indocyanine green with excitation light having a wavelength of 750 to 810 nm (nanometers), the indocyanine green emits fluorescence in an infrared region having a peak wavelength of 845 nm. The fluorescence is imaged by a camera capable of detecting infrared light, and the resulting image is displayed on a display unit such as a liquid crystal display panel. The near-infrared fluorescence imaging makes it possible to observe objects such as blood vessels and lymphatic vessels present at a depth of approximately 20 mm or less from the body surface.

Patent Literature 1 discloses a data collection method that compares a near-infrared fluorescence intensity distribution image obtained by irradiating a suspected body organ administered with indocyanine green with excitation light for indocyanine green and a cancer lesion distribution image obtained by making X-rays, nuclear magnetic resonance, or an ultrasonic wave act on the suspected organ before the indocyanine green administration with each other, and as cancer sub-lesion region data, collects data on a region that is detected in the near-infrared fluorescence intensity distribution image but is not detected in the cancer lesion distribution image.

### CITATION LIST

### Patent literature

### Patent Literature 1

International Patent Publication No. 2009/139466

### SUMMARY OF INVENTION

### Technical Problem

For example, when performing breast cancer surgery in breast surgery, it is necessary to identify the position of a sentinel lymph node. The sentinel lymph node is a lymph node that is first reached by cancer cells via lymph flow. If no cancer cells are found in the sentinel lymph node, it can be determined that the breast cancer has not metastasized to any other lymph node.

Meanwhile, indocyanine green used for the near-infrared fluorescence imaging has small molecular weight and high fluidity in a lymphatic vessel, and is therefore characterized by diffusing in a short period of time. For this reason, there occurs a problem that after time has passed since indocyanine green was administered, the whole of a lymphatic vessel emits fluorescence, and it becomes difficult to identify which lymph node is a sentinel lymph node. Such a problem of becoming more difficult to identify an affected area as time passes is a problem generally occurring not only when performing breast cancer surgery but when performing other near-infrared fluorescence imaging.

This invention is made in order to solve the above-described problem, and intends to provide an infrared light imaging apparatus that even after time has passed since a fluorescent dye was injected, can easily recognize an affected area.

### Solution to Problem

A first aspect of the present invention includes: an infrared light source adapted to irradiate a subject with an infrared ray for exciting a fluorescent dye injected into the subject; a camera that is for imaging infrared light emitted from the fluorescent dye by irradiating the fluorescent dye with the infrared ray and capable of detecting the infrared light; an image storage unit for storing an infrared image of the subject imaged by the camera; and an image processing unit adapted to simultaneously display a current infrared image, and a past infrared image stored in the image storage unit on a display unit.

A second aspect of the present invention includes: an infrared light source adapted to irradiate a subject with an infrared ray for exciting a fluorescent dye injected into the subject; a visible light source adapted to irradiate the subject with visible light; a camera that is for imaging infrared light emitted from the fluorescent dye by irradiating the fluorescent dye with the infrared ray, and visible light reflected on a surface of the subject, and capable of detecting the infrared light and the visible light; an image storage unit for storing an infrared image of the subject imaged by the camera; and an image processing unit adapted to simultaneously display a current visible image, and a past infrared image stored in the image storage unit on a display unit.

A third aspect of the present invention includes: an infrared light source adapted to irradiate a subject with an infrared ray for exciting a fluorescent dye injected into the subject; a visible light source adapted to irradiate the subject with visible light; a camera that is for imaging infrared light emitted from the fluorescent dye by irradiating the fluorescent dye with the infrared ray, and visible light reflected on a surface of the subject, and capable of detecting the infrared light and the visible light; a fusion unit adapted to prepare a fusion image by fusing together an infrared image and a visible image of the subject imaged by the camera; an image storage unit for storing the infrared image and the visible image of the subject imaged by the camera, or storing the fusion image that is prepared by the fusion unit with use of the infrared image and the visible image of the subject imaged by the camera; an image processing unit adapted to simultaneously display any one of a current infrared image, a current fusion image, and a current visible image, and one of a past infrared image based on an image stored in the image storage unit and a past fusion image based on an image stored in the image storage unit on a display unit.

A fourth aspect of the present invention is that the image processing unit displays a current image as a moving image, and also displays a past image as a still image.

A fifth aspect of the present invention is that the subject is breast cancer, and the image processing unit displays a past image of a sentinel lymph node on the display unit.

### Advantageous Effects of Invention

According to the first aspect of the present invention, by simultaneously displaying the current infrared image and the past infrared image on the display unit, even after time has passed since the fluorescent dye was injected, a specific part of the subject can be easily recognized.

According to the second aspect of the present invention, by simultaneously displaying the current visible image and the past infrared image on the display unit, even after time has passed since the fluorescent dye was injected, a specific part of the subject can be easily recognized.

According to the third aspect of the present invention, (1) by simultaneously displaying the current fusion image and the past fusion image on the display unit, (2) by simultaneously displaying the current fusion image and the past infrared image on the display unit, (3) by simultaneously displaying the current infrared image and the past fusion image on the display unit, (4) by simultaneously displaying the current infrared image and the past infrared image on the display unit, (5) by simultaneously displaying the current visible image and the past infrared image on the display unit, or (6) by simultaneously displaying the current visible image and the past fusion image on the display unit, even after time has passed since the fluorescent dye was injected, a specific part of the subject can be easily recognized.

According to the fourth aspect of the present invention, by displaying the current image as a moving image, the current state of the subject can be accurately recognized in real time.

According to the fifth aspect of the present invention, it is possible to easily recognize a sentinel lymph node from among a number of lymph nodes.

### BRIEF DESCRIPTION OF DRAWINGS

FIG. 1 is a schematic diagram of an infrared light imaging apparatus according to this invention.

FIG. 2 is a perspective view of an illumination/imaging unit 12.

FIG. 3 is a block diagram illustrating the main control system of the infrared light imaging apparatus according to this invention.

FIG. 4 is a schematic diagram illustrating images displayed on display units 14 and 15, respectively.

FIG. 5 is a schematic diagram illustrating images displayed on the display units 14 and 15, respectively.

FIG. 6 is a schematic diagram illustrating images displayed on the display units 14 and 15, respectively.

FIG. 7 is a schematic diagram illustrating images displayed on the display units 14 and 15, respectively.

FIG. 8 is a schematic diagram illustrating images displayed on the display units 14 and 15.

### DESCRIPTION OF EMBODIMENTS

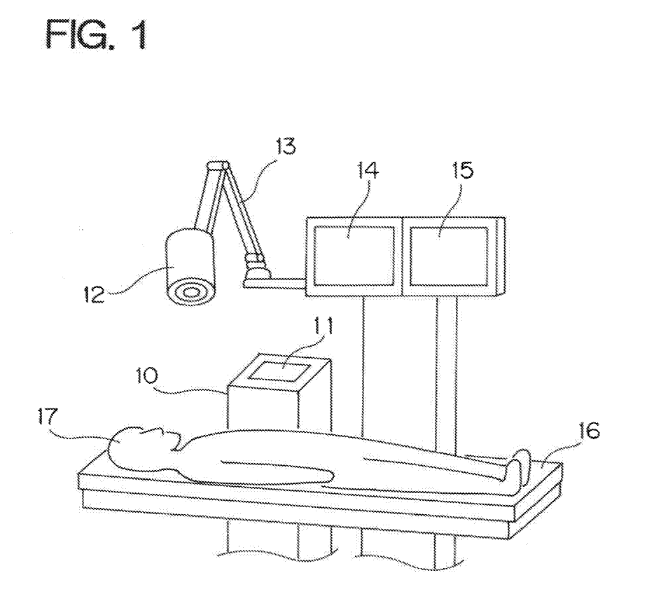

In the following, embodiments of this invention will be described on the basis of the drawings. FIG. 1 is a schematic diagram of an infrared light imaging apparatus according to this invention.

This infrared light imaging apparatus includes an input unit 11 such as a touch panel, and further includes a main body 10 incorporating the below-described controller 30 and the like, an illumination/imaging unit 12 movably supported by an arm 13, a pair of display units 14 and 15 each configured to include a liquid crystal display panel and the like, and a treatment table 16 on which a patient 17 is placed. Note that the illumination/imaging unit 12 is not limited to one supported by the arm 13, but may be one hand-held by an operator.

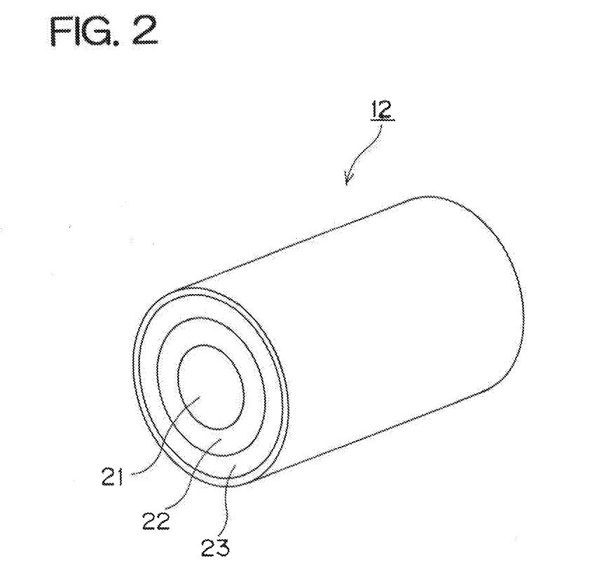

FIG. 2 is a perspective view of the above-described illumination/imaging unit 12.

The illumination/imaging unit 12 includes: a camera 21 that can detect infrared rays and visible light; an infrared light source 22 that is disposed in the outer circumferential part of the camera 21; and a visible light source 23 that is disposed in the outer circumferential part of the infrared light source 22. Note that in the case of displaying only an infrared image, it is not necessarily required to include the visible light source 23.

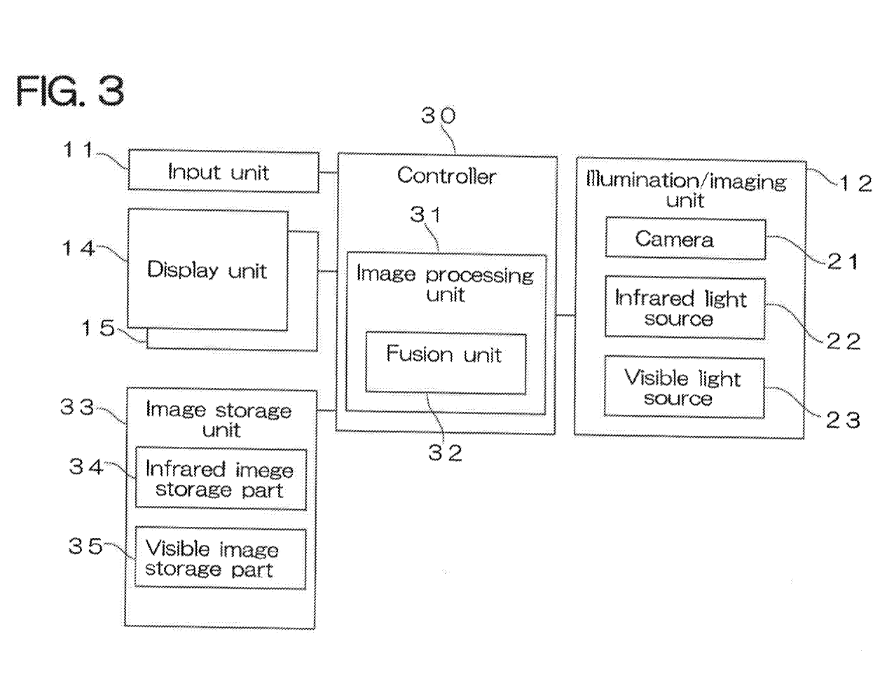

FIG. 3 is a block diagram illustrating the main control system of the infrared light imaging apparatus according to this invention.

The infrared light imaging apparatus is configured to include: a CPU that performs logical operations; a ROM that stores an action program necessary to control the apparatus; a RAM that temporarily stores data or the like during the control, and the like, and includes the controller 30 adapted to control the whole of the apparatus. As described below, the controller 30 includes an image processing unit 31 having a fusion unit 32. Also, the controller 30 is connected to the above-described input unit 11 and display units 14 and 15. Further, the controller 30 is connected to the illumination/imaging unit 12 including the camera 21, infrared light source 22, and visible light source 23. Still further, the controller 30 is also connected to an image storage unit 33 adapted to store images imaged by the camera 21. The image storage unit 33 is configured to include an infrared image storage part 34 adapted to store infrared images and a visible image storage part 35 adapted to store visible images. Note that in place of providing the infrared image storage part 34 and the visible image storage part 35, it is also possible to provide a fusion image storage part adapted to store images each formed by fusing a visible image and an infrared image together.

In the following, actions performed when performing surgery using the infrared light imaging apparatus according to this invention will be described. Note that in the following, a description on the case where breast cancer surgery is performed on the patient 17 will be given.

When performing the breast cancer surgery using the infrared light imaging apparatus according to this invention, indocyanine green is injected by a syringe into an appropriate breast of the patient 17 lying on her/his back on the treatment table 16. After that, a subject including an affected area is irradiated with the infrared rays from the infrared light source 22 as well as being irradiated with the visible light from the visible light source 23. Note that as the infrared rays, near-infrared light having a wavelength of 750 to 850 nm and acting as excitation light for indocyanine green to emit fluorescence is employed. This causes the indocyanine green to emit fluorescence in an infrared region having a peak wavelength of 845 nm.

Then, the affected area and its surroundings of the patient 17 are imaged by the camera 21. The camera 21 is capable of detecting infrared light and visible light. An infrared image and a visible image imaged by the camera 21 are sent to the image processing unit 31 illustrated in FIG. 3. In the image processing unit 31, the infrared image and the visible image are respectively converted into pieces of image data displayable on the display units 14 and 15. The infrared image data is stored in the infrared image storage part 34 of the image storage unit 33. On the other hand, the visible image data is stored in the visible image storage part 35 of the image storage unit 33.

Also, the fusion unit 32 of the image processing unit 31 uses the infrared image data and the visible image data to prepare a fusion image formed by fusing the visible image and the infrared image together. Then, the image processing unit 31 selectively displays the infrared image, visible image, and fusion image on the display units 14 and 15.

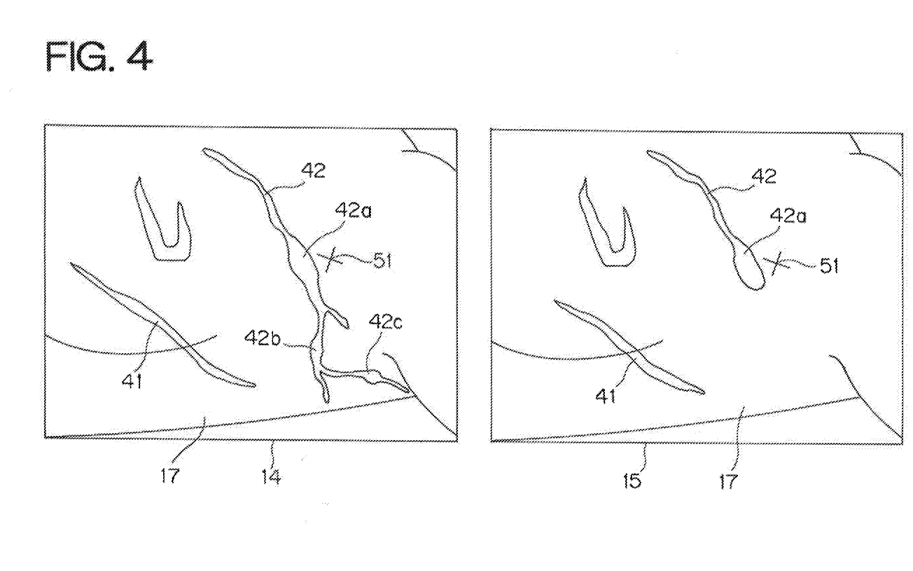

FIGS. 4 to 8 are schematic diagrams each illustrating images displayed on the display units 14 and 15.

FIG. 4 illustrates a first embodiment of this invention. In this embodiment, on the display unit 14, a current fusion image of the affected area and its surroundings of the patient 17 is displayed as a moving image, whereas on the display unit 15, a past fusion image of the affected area and its surroundings of the patient 17 is displayed as a still image. That is, the image processing unit 31 takes in an infrared image and visible image imaged by the camera 21 to make the fusion unit 32 fuse them together, and displays the current fusion image of the affected area and its surroundings of the patient 17 on the display unit 14 as a moving image. Also, the image processing unit 31 reads a past infrared image stored in the infrared image storage part 34 and a past visible image stored in the visible image storage part 35 to make the fusion unit 32 fuse them together, and displays the past fusion image of the affected area and its surroundings of the patient 17 on the display unit 14 as a still image. Note that in the case where the past fusion image is stored in the fusion image storage part, it is only necessary to read the past fusion image from the fusion image storage part to display it on the display unit 14.

As illustrated in FIG. 4, in the fusion image displayed on the display unit 14 as a moving image, lymphatic vessels 41 and 42 are indicated by the fluorescence emitted from the indocyanine green. In addition, reference sign 51 in FIG. 4 denotes a mark placed on the body surface of the patient 17 for the surgery. As illustrated in FIG. 4, the lymphatic vessel 42 includes multiple lymph nodes 42a, 42b, and 42c. Among them, the lymph node 42a first reached by lymph from the breast is a sentinel lymph node.

In the breast cancer surgery, it is necessary to identify the sentinel lymph node 42a; however, the indocyanine green has small molecular weight and high fluidity in the lymphatic vessel 42, and is therefore characterized by diffusing in a short period of time. For this reason, after time has passed since the indocyanine green was administered, the whole of the lymphatic vessel 42 emits the fluorescence, and it becomes difficult to identify where the sentinel lymph node 42a is present in the lymphatic vessel 42 or identify which lymph node is the sentinel lymph node 42a.

Therefore, on the display unit 15, the past fusion image of the affected area and its surroundings of the patient 17 is displayed as a still image. The past fusion image of the affected area and its surroundings of the patient 17 is stored together with the time when imaging was actually performed and the time when the indocyanine green was injected. Alternately, while checking the moving fusion image displayed on the display unit 14, the operator gives a time stamp to the fusion image. Then, using those times or the time stamp, an image at the time when the indocyanine green flowing through the lymphatic vessel 42 reached the sentinel lymph node 42a as the first lymph node is identified, and a fusion image at the time is displayed on the display unit 15 as a still image. This makes it possible to easily identify the position of the sentinel lymph node 42a.

The operator identifies the sentinel lymph node 42a, which is not viewable even if visually observing the patient 17, by checking the current fusion image displayed on the display unit 14, and performs surgery for removing it. When doing this, on the basis of the past fusion image displayed on the display unit 15 as a still image, it can be identified which fluorescent part is the sentinel lymph node 42a. The removed sentinel lymph node 42a is subjected to pathological examination. If the cancer does not metastasize to the sentinel lymph node 42a, the surgery is completed only by removing the breast cancer. On the other hand, if the cancer has metastasized to the sentinel lymph node 42a, surgery for removing the whole of the lymphatic vessel is performed.

As described, since the infrared light imaging apparatus according to this invention displays the current fusion image of the affected area and its surroundings of the patient 17 on the display unit 14 as a moving image, as well as displaying the past fusion image of the affected area and its surroundings of the patient 17 on the display unit 15 as a still image, it is possible to easily identify the position of the sentinel lymph node 42a during the surgery, and therefore the surgery to remove the sentinel lymph node 42a can be appropriately performed.

In the embodiment illustrated in FIG. 4, the current infrared image and visible images are displayed fused together on the display unit 14 as a moving image based on the fusion image, whereas the past infrared image and visible image are displayed fused together on the display unit 15 as a still image based on the fusion image. However, it may be adapted to display the infrared images or the visible images on the display units 14 and 15. In the following, embodiments in such a case will be described.

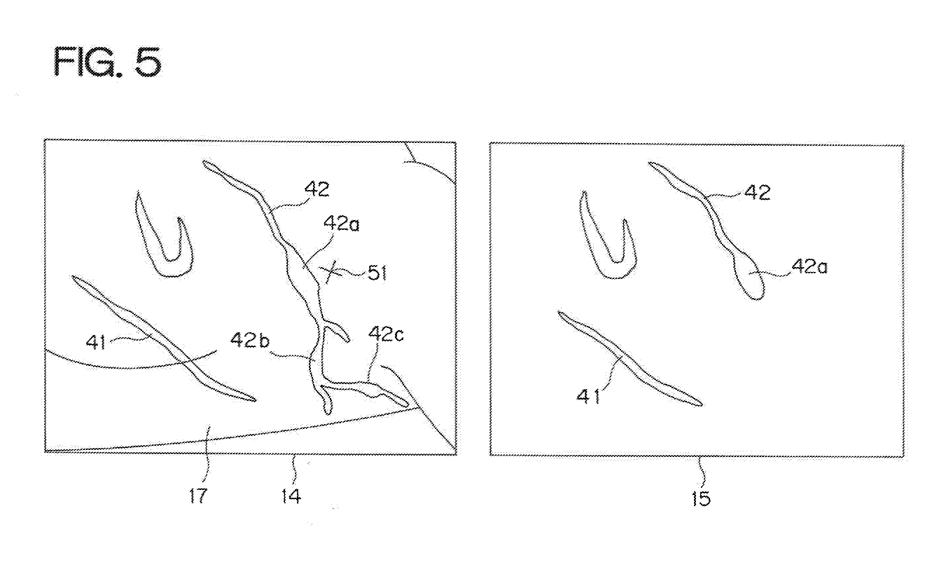

FIG. 5 illustrates a second embodiment of this invention. In this embodiment, on the display unit 14, the current fusion image of the affected area and its surroundings of the patient 17 is displayed as a moving image, whereas on the display unit 15, the past infrared image of the affected area and its surroundings of the patient 17 is displayed as a still image. Even in such an embodiment, it is possible to easily identify the position of the sentinel lymph node 42a during the surgery, and therefore the surgery to remove the sentinel lymph node 42a can be appropriately performed.

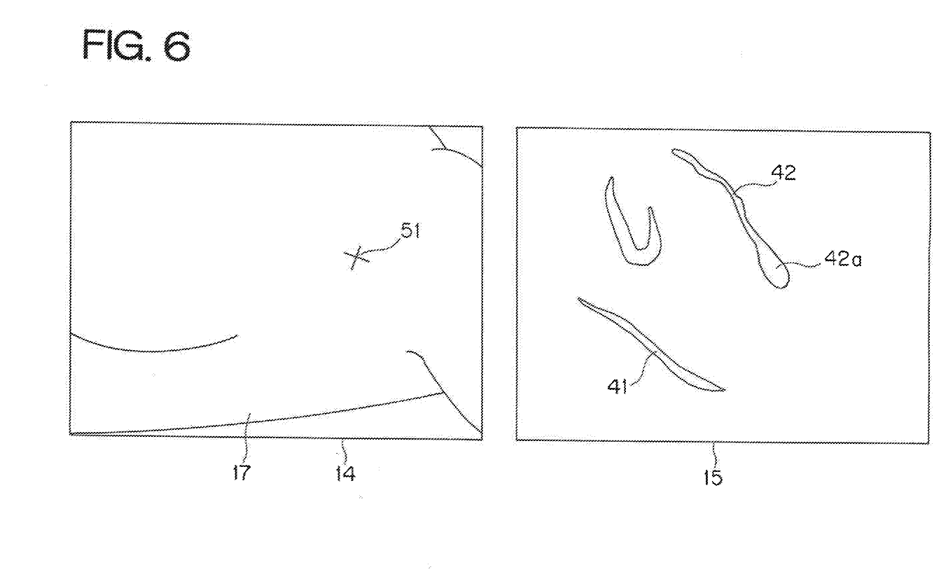

FIG. 6 illustrates a third embodiment of this invention. In this embodiment, on the display unit 14, the current visible image of the affected area and its surroundings of the patient 17 is displayed as a moving image, whereas on the display unit 15, the past infrared image of the affected area and its surroundings of the patient 17 is displayed as a still image. In such an embodiment, the visible image makes it possible to establish and easily recognize the relationship between the patient 17 in an actual state and the visible image displayed on the display unit 14.

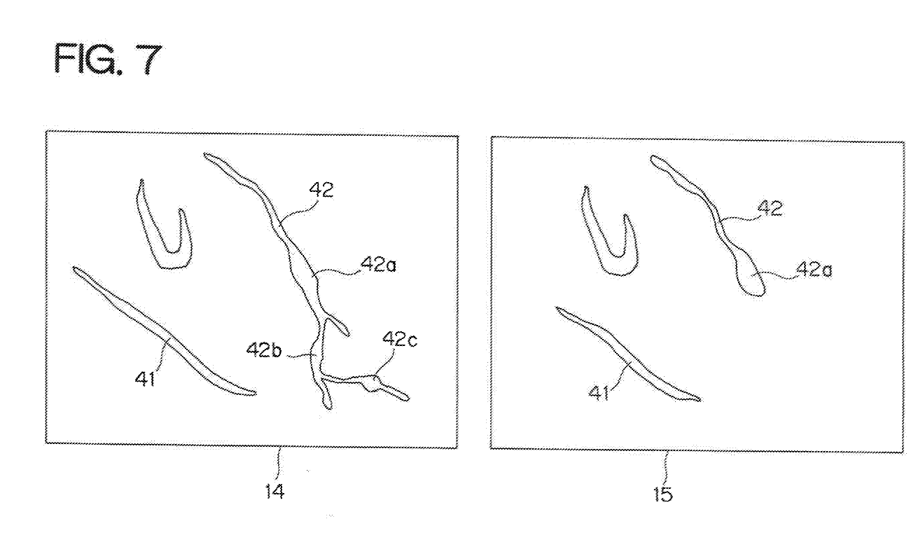

FIG. 7 illustrates a fourth embodiment of this invention. In this embodiment, on the display unit 14, the current infrared image of the affected area and its surroundings of the patient 17 is displayed as a moving image, whereas on the display unit 15, the past infrared image of the affected area and its surroundings of the patient 17 is displayed as a still image. In such an embodiment, by comparing the infrared images with each other, it becomes possible to more easily identify the sentinel lymph node 42a from among the multiple lymph nodes 42a, 42b, and 42c.

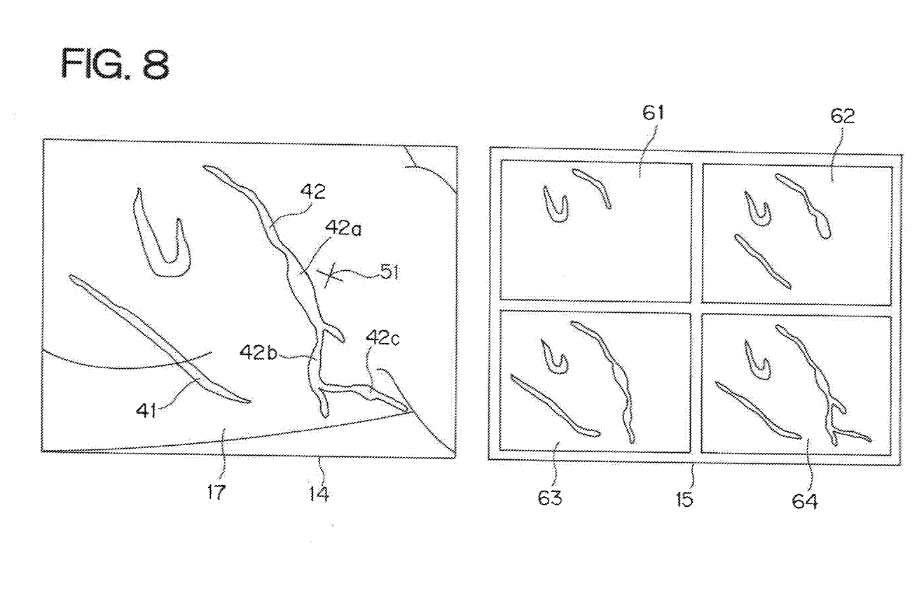

FIG. 8 illustrates a fifth embodiment of this invention. In this embodiment, on the display unit 14, the current fusion image of the affected area and its surroundings of the patient 17 is displayed as a moving image, whereas on the display unit 15, multiple past infrared images 61, 62, 63, and 64 of the affected area and its surroundings of the patient 17 are displayed arranged with time and reduced as still images. In such an embodiment, by comparing the multiple past infrared images 61, 62, 63, and 64, it becomes possible to easily identify the sentinel lymph node 42a.

That is, in any of the embodiments illustrated in FIGS. 5 to 8, as in the embodiment illustrated in FIG. 4, it is possible to easily identify the position of the sentinel lymph node 42a during the surgery, and therefore the surgery to remove the sentinel lymph node 42a can be appropriately performed.

Note that in any of the above-described embodiments, the display unit 14 displays a moving image, whereas the display unit 15 displays a still image. However, as necessary, it may be adapted to selectively display a moving image and a still image. In addition, on the display unit 14, the current infrared image of the affected area and its surroundings of the patient 17 may be displayed as a moving image, whereas on the display unit 15, the past fusion image of the affected area and its surroundings of the patient 17 may be displayed as a still image. Alternatively, on the display unit 14, the current visible image of the affected area and its surroundings of the patient 17 may be displayed as a moving image, whereas on the display unit 15, the past fusion image of the affected area and its surroundings of the patient 17 may be displayed as a still image. Further, it may be adapted to simultaneously display all of the infrared images, the visible images, and the fusion images.

Also, in any of the above-described embodiments, the infrared images, the visible images, and the fusion images are selectively displayed on the pair of display units 14 and 15; however, it may be adapted to use a relatively large-sized single display unit to separately display multiple images on the display unit, or use three or more display units to selectively display the infrared images, the visible images, and the fusion images on these display units.

Further, in any of the above-described embodiments, as the infrared light acting as excitation light, the near-infrared light having a wavelength of 750 to 850 nm is used; however, it may be adapted to use other infrared light.

Still further, in any of the above-described embodiments, the case where indocyanine green is used as a fluorescent dye is described; however, another fluorescent dye may be used. In some cases such as the case of using 5-aminolevulinic acid (5-ALA) to make a neoplasm part emit fluorescence for display, this invention may be applied.

For descriptive convenience, in any of the above-described embodiments, the case of imaging the affected area is taken as an example to give the description; however, it is also possible to use the present invention to perform work such as to make the observation of blood flow in a normal area. Accordingly, the affected area and the normal area imaged in accordance with the present invention are collectively defined as a subject.

### REFERENCE SIGNS LIST

10 Main body
11 Input unit
12 Illumination/imaging unit
13 Arm
14 Display unit
15 Display unit
16 Treatment table
17 Patient
21 Camera
22 Infrared light source
23 Visible light source
30 Controller
31 Image processing unit
32 Fusion unit
33 Image storage unit
34 Infrared image storage part
35 Visible image storage part

1. An infrared light imaging apparatus comprising:
an infrared light source adapted to irradiate a subject with an infrared ray for exciting a fluorescent dye injected into the subject;
a camera that is for imaging infrared light emitted from the fluorescent dye by irradiating the fluorescent dye with the infrared ray and capable of detecting the infrared light;
an image storage unit for storing an infrared image of the subject imaged by the camera; and
an image processing unit adapted to simultaneously display a current infrared image, and a past infrared image stored in the image storage unit on a display unit.

2. An infrared light imaging apparatus comprising:
an infrared light source adapted to irradiate a subject with an infrared ray for exciting a fluorescent dye injected into the subject;
a visible light source adapted to irradiate the subject with visible light;
a camera that is for imaging infrared light emitted from the fluorescent dye by irradiating the fluorescent dye with the infrared ray, and visible light reflected on a surface of the subject, and capable of detecting the infrared light and the visible light;
an image storage unit for storing an infrared image of the subject imaged by the camera; and
an image processing unit adapted to simultaneously display a current visible image, and a past infrared image stored in the image storage unit on a display unit.

3. An infrared light imaging apparatus comprising:
an infrared light source adapted to irradiate a subject with an infrared ray for exciting a fluorescent dye injected into the subject;
a visible light source adapted to irradiate the subject with visible light;
a camera that is for imaging infrared light emitted from the fluorescent dye by irradiating the fluorescent dye with the infrared ray, and visible light reflected on a surface of the subject, and capable of detecting the infrared light and the visible light;
a fusion unit adapted to prepare a fusion image by fusing together an infrared image and a visible image of the subject imaged by the camera;
an image storage unit for storing the infrared image and the visible image of the subject imaged by the camera, or storing the fusion image that is prepared by the fusion unit with use of the infrared image and the visible image of the subject imaged by the camera; and
an image processing unit adapted to simultaneously display any one of a current infrared image, a current fusion image, and a current visible image, and one of a past infrared image based on an image stored in the image storage unit and a past fusion image based on an image stored in the image storage unit on a display unit.

4. The infrared light imaging apparatus according to claim 1, wherein
the image processing unit displays a current image as a moving image, and also displays a past image as a still image.

5. The infrared light imaging apparatus according to claim 1, wherein
the subject is breast cancer, and
the image processing unit displays a past image of a sentinel lymph node on the display unit.
